(19) United States
(12) Patent Application Publication
(10) Pub. No.: US 2012/0100192 A1
(43) Pub. Date: Apr. 26, 2012

(54) LOCAL THERAPEUTIC RELEASE DEVICE

(75) Inventors: Adel Penhasi, Holon (IL); Albert Reuveni, Jerusalem (IL); Eyal Shoshani, Jerusalem (IL)

(73) Assignee: DEXCEL PHARMA TECHNOLOGIES LTD., OrAkiva (IL)

(21) Appl. No.: 13/380,643

(22) PCT Filed: Jun. 24, 2010
(86) PCT No.: PCT/IL2010/000509

§ 371 Date: Dec. 23, 2011

Related U.S. Application Data
(60) Provisional application No. 61/222,203, filed on Jul. 1, 2009.

(51) Int. Cl. A61K 9/00; A61P 1/02; A61K 31/192

(52) U.S. Cl. 424/400; 514/570

(57) ABSTRACT

An oral delivery device for the treatment of periodontal disease, the device being in a solid unit dosage form configured for insertion into a periodontal pocket of a patient. The device consists of: (a) a biodegradable pharmaceutically acceptable water-insoluble polymer in the form of a matrix; (b) a therapeutically effective amount of at least one anti-inflammatory agent dispersed within the matrix; (c) optionally a plasticizing agent; (d) optionally at least one of a wetting agent, a suspending agent and a dispersing agent; and (e) optionally an enzymatically biodegradable pharmaceutically acceptable water soluble polymer dispersed within the matrix. The biodegradable water-insoluble polymer is degradable by enzymatic degradation, physical disintegration or a combination thereof. Also disclosed is a periodontal implant comprising the device and a method for the treatment of periodontal disease comprising administering to a periodontal pocket of a patient in need of such treatment the delivery device.

### FIELD OF THE INVENTION

This invention relates to local therapeutic-release compositions, suitable for achieving the local therapeutic release of anti-inflammatory drugs. The invention also pertains to a method of use of this composition in the periodontal pocket for the treatment of periodontal disease.

### BACKGROUND OF THE INVENTION

The two major diseases of the oral cavity are dental caries, a disease process by which cavities are produced in the tooth surface, and periodontal disease, a process in which the bone and soft tissues supporting the tooth are destroyed. Periodontal diseases are a very common occurrence affecting, at a conservative estimate, between 70%-90% of the world population and is the major cause of tooth loss in people over 35 years of age. Periodontal disease is an all-inclusive term for a variety of clinical conditions that are forms of either gingivitis or periodontitis. Gingivitis is an inflammation of the gingiva (or gums) that can be associated with poor oral hygiene and/or the hormonal state of the patient. It is believed that gingivitis, if untreated, will develop into periodontitis. Periodontitis is a bacterial disease in which the infection has progressed to involve the oral tissues which retain the teeth in the jawbone. Periodontitis, if untreated, will eventually result in the loss of the affected tooth. Chronic periodontitis is characterized by resorption of the alveolar bone as well as loss of soft tissue attachment to the tooth in adults.

Although dental caries may be effectively treated with a combination of proper hygiene and fluoride, periodontal disease is often more refractile to treatment. This difference in amenability to treatment reflects the markedly different environments of the oral and periodontal cavities. The oral cavity is essentially an aerobic environment, which is perfused by saliva. In contrast, the periodontal microenvironment is more anaerobic and is perfused by a plasma filtrate, known as the “gingival crevicular fluid”. The growth of microorganisms within this microenvironment has been shown to be the cause of periodontal disease. Hence, the treatment of the disease is directed toward controlling this growth. As the periodontal disease becomes more established, the periodontal microenvironment becomes more anaerobic and the flow of gingival crevice fluid increases. [An excellent review of periodontal disease, and the methods for its treatment, is provided by Goodson J. M. (In: Medical Applications of Controlled Release, Vol. II, Applications Evaluation (Langer, R. S., et al., Eds.), CRC Press, Inc., Boca Raton, Fla. (1984), pp. 115-138), which is incorporated by reference herein].

Efforts to treat periodontal disease have been impeded by several factors. Because the site of the bacterial infections is largely inaccessible to agents present in the oral cavity, antimicrobial agents provided to the oral cavity are generally ineffective. The increased flow of gingival crevice fluid, which accompanies periodontal disease, has the effect of diluting and removing therapeutic agents placed within the periodontal crevice. Deviceic administration of antibiotics has been shown to be a useful method of controlling the subgingival flora, however discontinuation of therapy is often associated with the return of the potential pathogens to the pockets. Deviceic administration, therefore, has had only variable success in treating periodontal disease. Long-term antibacterial therapy has been used, but the potential dangers associated with this form of treatment, which include the development of resistant strains and super-imposed infections, do not warrant its serious consideration. Antibacterial agents such as chlorhexidine and quaternary ammonium salts in the form of mouth rinses have proved to be successful in preventing periodontal disease. These agents, however, are unable to affect the subgingival flora when administered in this form as they do not penetrate into the pockets which are the result of the disease. Hence, they cannot be used in mouth rinses to treat an established periodontal disease. Patient acceptance has significantly limited the utility of non-pharmacological treatments of periodontal disease. The most widely used non-pharmacological approach to date has been mechanical cleaning methods combined with surgery. Although this method has proved to be fairly successful in treating individuals, there is still a high recurrence rate. There is also the problem of motivating people to good oral hygiene habits that they will maintain throughout their lives.

In response to the importance of treating periodontal disease, and the failure of conventional control therapies, researchers have developed control-release pharmaceutical compositions which are capable of being inserted into the periodontal cavity and of slowly releasing an antimicrobial agent. Goodson et al. (J. Clin. Periodon. 6:83 (1979); J. Periodont. Supp.-Special Issue 81-87 (1985)) proposed the use of a device that would provide a sustained release of antibacterial agents to control the pocket flora. The most investigated devices for controlled release comprise incorporating such a drug into a polymeric matrix, which is then shaped into a convenient form and implanted into the periodontal cavity. Goodson J. M. (U.S. Pat. Nos. 4,764,377 and 4,892,736) discloses the incorporation of tetracycline into non-degradable polymeric fibers which can be wrapped around the tooth and release the antibiotic into the periodontal cavity for several days. The fibers needed to be fastened in place with an adhesive and need to be removed at the end of the treatment period.

Ethyl cellulose has been successfully employed as a polymeric matrix of a periodontal implant. Various antibacterial agents, such as chlorhexidine, metronidazole, iodine and cetyl pyridinium chloride, have been incorporated into such ethyl cellulose films. Loesche, W. J. (U.S. Pat. No. 4,568,535) discloses the use of periodontal implants composed of ethyl cellulose which contain metronidazole in the treatment of periodontal disease. Although such films were found to be effective in treating periodontal disease, their non-biodegradable nature required their removal after the conclusion of therapy.

Dunn, R. L. (U.S. Pat. No. 5,702,716) describes the incorporation of doxycycline into a gel that solidifies in the periodontal pocket. The antibiotic drug is released over several days. The solidified gel must be removed at the end of the treatment. Hence, a major therapeutic goal is the development of a biodegradable implant which would not need to be removed from the patient.

Degradable polymers and copolymers which have been substantially investigated as potential implant compositions include poly(lactic acid), poly(glycolic acid), and poly(lactic acid)-poly(glycolic acid) copolymer. The biodegradation of poly(lactic acid) and poly(glycolic acid) can require three to five months. Thus, it would not be preferable to employ implants composed of such polymers in situations where more rapid biodegradation is desired.

Absorbable periodontal implants have been described which used a hydroxypropylcellulose polymer. Suzuki, Y., et. al., (U.S. Pat. No. 4,569,837) discloses the use of water-soluble polymeric substances (such as methyl cellulose, gelatin, etc.) as a polymeric matrix for a periodontal implant. Lading, P. (U.S. Pat. No. 5,143,934) describes the incorporation of metronidazole into a gel that semi-solidifies in the periodontal pocket as a liquid crystalline formulation. The antibiotic drug is released over about one day as the gel dissolves in the gingival crevicular fluid.

A biodegradable sustained-release composition has been described by Freidman, M. et al., (U.S. Pat. No. 5,023,769) which is capable of delivering a pharmacological composition for a period of time sufficient to treat a periodontal infection. The pharmacological agent (chlorhexidine antiseptic) comprises a polymeric matrix containing a plasticizing agent, and the active agent, wherein the polymeric matrix comprises a cross-linked, water-insoluble protein formed from a water soluble protein.

The compositions described above have varying efficacy in reducing the bacterial load of the periodontal pocket and in reducing pocket depth and gingival level of attachment. None of the above mentioned formulations are particularly efficacious in causing alveolar bone regrowth or even in arresting alveolar bone resorption.

One of the drugs that is known in its ability to reduce periodontal pocket depth or alveolar bone resorption is flurbiprofen (FBP). FBP is a non-steroidal anti-inflammatory drug (NSAID) which also exhibits analgesic and anti-pyretic activity. Flurbiprofen inhibits prostaglandin synthesis by inhibition of cyclooxygenase, an enzyme that catalyses the formation of prostaglandin precursors from arachidonic acid. Wechter, W. J. (European patent No. 137,668 B1) suggests the use of FBP for the treatment of bone resorption and the inducing of bone growth.

Williams et al (J. Perio. Res. 19:633-637, 1984; 22:403-407, 1987; 23:166-169, 1988) and Jeffcoat et al (J. Perio. Res. 21:624-633, 1986) demonstrated that devices and topical application of FBP to beagle dogs for 6-12 months inhibited alveolar bone loss in naturally occurring periodontitis. Offenbacher et al (J. Perio. Res. 22:473-481, 1987) demonstrated that FBP administered deviceatically to Macaca mulatta monkeys with experimentally induced periodontal disease resulted in significant inhibition of attachment, bleeding on probing and gingival redness. Chung et al (J. Perio. Res. 32:172-175, 1997) tested drug (FBP and others)-loaded biodegradable membrane for guided bone regeneration (GBR). The loaded membrane was effective for osteoid tissue and new bone formation in the bony defect prepared in rat calvaria to compare with that by unloaded membrane. The successful results seen in animal models treated with FBF led to the conclusion that clinical studies could be performed in patients with moderate to severe periodontal disease.

Jeffcoat et al (J. Perio. Res. 23:381-385, 1988) were the first investigators who demonstrated the clinical effects of FBP on the progression of periodontal disease. As evidenced by standardized radiography and reduced radiopharmaceutical uptake, treatment with FBP (100 mg/day) for two months increased bone metabolism. A study for 24 months using FBP by Williams et al (J. Dental Res. 70:468, 1991) found that the FBP-treated patient group showed reduction in bone loss. This demonstrated that FBP treatment can be a significant inhibitor of alveolar bone loss. Heasman et al (J. Clin. Periodontol, 20:457-464, 1993) examined the effect of FBP given topically (toothpaste, 1% w/w) twice daily for 12 months to patients with periodontal disease. The, FBP treated group showed statistically significant bone gain. This suggests that the topical application of FBP may have a positive bone gain effect in humans.

Dimani, N. C. (U.S. Pat. No. 5,447,725) suggests a delivery device that hardens on contact with the periodontal tissue after a solvent is leached out and that releases FBP or other drugs in the periodontal pocket. The material is inserted into the periodontal pocket as a gel from a syringe and hardens in situ. Syringing an exact dose of a gel into a body crevice such as a periodontal pocket and having a known dose of the drug solidifying therein is difficult to carry out and difficult to control.

Friedman et al (U.S. Pat. No. 5,023,082) discloses biodegradable sustained-release liquid compositions capable of achieving the sustained release of a pharmaceutical agent such as an anti-inflammatory agent. The liquid precursor compositions can be formed into solid implant devices after administration which may be used to treat diseases such as periodontal disease which require prolonged drug release.

Friedman et al (U.S. Pat. No. 5,160,737) discloses a liquid methacrylic acid copolymer composition that contains a release adjusting agent and a pharmacological agent. The composition forms a solid film upon drying, and is capable of accomplishing the sustained release of the pharmacological agent such as to permit its use in the treatment or prevention of dental or dermatological conditions.

Lerner et al (U.S. Pat. No. 6,197,331) discloses a controlled-release solid composition for the oral cavity or “pharmaceutical oral patch” that adheres to hard dental surfaces, such as teeth and dentures, and releases an active pharmaceutical agent into the oral cavity. Release of the agent is for a predetermined period of time and at a predetermined sustained concentration. The site of action of the agent is local or deviceic.

Uhrich et al (U.S. Pat. No. 6,685,928) discloses methods of promoting healing through enhanced regeneration of tissue (e.g. hard tissue or soft tissue) by contacting the tissue or the surrounding tissue with an anti-inflammatory agent in a carrier comprising aromatic polyanhydrides. These methods are useful in a variety of dental and orthopedic applications.

Penhasi et al (U.S. Patent Application No. 2004/0185009) discloses an oral delivery device for the treatment of periodontal disease, being in a solid unit dosage form for administration to a patient and comprising: (i) a biodegradable or bioerodible pharmaceutically acceptable polymer; (ii) a therapeutically effective amount of at least one antibacterial agent; and (iii) a therapeutically effective amount of at least one anti-inflammatory agent, the relative weight ratio between the antibacterial agent and the anti-inflammatory agent ranging from about 7:1 to about 1:5. The device may further comprise at least one of a cross-linking agent, a plasticizing agent, a wetting agent, a suspending agent, a surfactant and a dispersing agent.

### SUMMARY OF THE INVENTION

In a first aspect, the present invention relates to an oral delivery device for the treatment of periodontal disease, said device being in a solid unit dosage form configured for insertion into a periodontal pocket of a patient, consisting of:
(a) a biodegradable pharmaceutically acceptable water-insoluble polymer in the form of a matrix;
(b) a therapeutically effective amount of at least one anti-inflammatory agent dispersed within the matrix;
(c) optionally a plasticizing agent;
(d) optionally at least one of a wetting agent, a suspending agent and a dispersing agent; and
(e) optionally an enzymatically biodegradable pharmaceutically acceptable water soluble polymer dispersed within the matrix,
   wherein said biodegradable water-insoluble polymer is degradable by enzymatic degradation, physical disintegration or a combination thereof.

In one embodiment, the physical disintegration is by hydration and swelling of the water-insoluble polymer. In another embodiment, the biodegradable water-insoluble polymer is not degradable by hydrolysis. In a further embodiment, the water-insoluble polymer is present at a concentration of from about 20% to about 70%.

A further aspect of the invention is a periodontal implant comprising the device of the invention.

A still further aspect of the invention is a method for the treatment of periodontal disease comprising administering to a periodontal pocket of a patient in need of such treatment the delivery device of the invention.

One embodiment of the delivery device that would be most advantageous would be one that has an exact dose of drug predetermined, is easy to insert, is retained in a periodontal pocket without the need of adhesives to keep it from falling out, gives sustained release of the anti-inflammatory drug over several days, and biodegrades so that there is no need for the removal of the device after the treatment period. Ease of insertion and dose control can be obtained by having the delivery device preformed into a rigid thin film that easily slips into a crevice such as a periodontal pocket with the aid of a simple tweezers. The adherence of the dosage form to the inside of the pocket is obtained by the drug delivery device softening and swelling, thereby adhering to the inside of the pocket.

The precursor solutions to drug delivery devices of this invention are used to form drug delivery devices that are polymeric solids that may be cast as films, pellets, granules, cylinders or any other convenient shape for the task at hand. The devices allow local delivery of the drug at the target site. The devices may be used as implants for the extended delivery of drug. The devices may also be used as inserts to body crevices as well as drug delivery devices in the body in general and, in one embodiment, in the oral cavity. Most preferentially, the devices may be used as an insert into periodontal crevices or pockets, or as an implant in periodontal surgery.

A drug delivery device for implantation in the body or insertion in a crevice in the body will preferentially be one that can target the drug to the organ desired, deliver the drug in a local fashion, and degrade in the body to harmless by-products so that the device need not be removed when it has finished its useful function. Preformed devices would negate the dose control problem. Both the in situ and preformed polymers of this sort tend to biodegrade slowly and are useful for delivery devices designed for prolonged delivery in the multi-week to months time frame. They do, however, biodegrade to amino acids which are biocompatible and non toxic. Poly amino acids and proteins have been found useful as the basis for drug delivery devices since their degradation products are harmless amino acids and their biodegradation is facile in many parts of the body.

Useful polymers for drug delivery include cross-linked water-soluble protein, cellulose or cellulose derivative, starch or starch derivative, glyceryl monostearate, carbomer, PVP (polyvinylpyrrolidone), gum, acacia gum, guar gum, polyvinyl alcohol, polyhydroxyethyl metacrylate, polyhydroxymethyl metacrylate polyacrylic acid, polyacryl amide and polyethylene glycols, an enzyme and fibrinogen. For example, proteins derived from connective tissue such as collagen and gelatin, and proteins of the albumin class that may be derived from milk, serum, or from vegetable sources may be used, with gelatin and hydrolyzed gelatin being the most preferable. In one embodiment, the hydrolyzed gelatin may have a molecular weight in the range of 1-20 K Dalton. Proteins, however, tend to be water soluble. In a soluble form the protein is less useful for sustained release of a drug since its solubilization will remove it from the body in too short a time. It is therefore desirable to render the protein water insoluble while maintaining its ability to biodegrade through normal enzymatic processes and permitting the release of the anti-inflammatory agent from the delivery device. This insolubilization of the protein may be done by making insoluble salts of the protein, insoluble complexes of the protein or most preferably by crosslinking the protein. In one embodiment, a water-soluble polymer is cross-linked by a curing process in the presence of a cross-linking agent, wherein said curing process is selected from the group consisting of heat, humidity, pressure, radiation, and the vapors of a cross-linking agent Since proteins in general contain lysine and arginine residues with amino reactive groups and serine, threonine and tyrosine with hydroxyl side chains, one preferable and well accepted method of crosslinking proteins is with aldehydes or dialdehydes. Formaldehyde, carbodiimide and more preferably glutaraldehyde are well known in the art as methods of crosslinking proteins. The crosslinked protein is rendered insoluble but its ability to be degraded by proteases in the body is maintained. The amount of crosslinking can be controlled by the ratio of the crosslinking agent to the protein side groups with which it is to react. The more heavily crosslinked the protein the less soluble it will be and the more slowly it will be biodegraded by protease enzymes. For example the most preferable amount of glutaraldehyde for crosslinking hydrolyzed gelatin has been found to be the amount that is stoicheometric with the amino side chains in the protein.

While for certain uses (e.g. the insertion of a depot of drug into the body where a crevice is not available) the insertion of liquid formulations may be easier than a preformed solid dosage form, in general a preformed solid dosage form is easier to handle and insert into an open crevice and gives better control of the drug dose. The incorporation of the drug in the delivery device must be uniform so as to keep tight control over the dosing level. If one chooses crosslinked proteins as the delivery device of choice because of its delivery, degradation, and non toxic by-product properties, one is faced with a problem of incorporating non water soluble drugs into such a device. While many methods exist to form homogeneous mixtures, the drug would not be incorporated into the matrix in a complete fashion. When all the components are dissolved in a solution the mixture of the components upon solidification is considerably more intimate and the control of the drug delivery from the crosslinked protein is much enhanced.

Many drugs that are not soluble to any extent in aqueous solutions are soluble in alcohol solutions. The alcohols useful with the aqueous solutions of the proteins are preferably ethanol, isopropanol and n-propanol, with ethanol being the most preferable. Proteins of low molecular weight and a relatively high proportion of hydrophobic side groups do not precipitate from aqueous solution when a certain proportion of alcohol is added. A preferable protein with regards to this property is hydrolyzed gelatin of number average molecular weight less than 20,000 and most preferably less than 13,000 but more than 1000. This protein is stable in solutions that contain over 50% ethanol allowing the incorporation of aqueous solutions of non water soluble drugs that are soluble in the alcohol.

A solid device for insertion into a body crevice needs to be rigid enough to be inserted against a certain amount of back pressure exhibited by the frictional forces on the device when being inserted, but pliable enough so as not to break and pliable enough to conform to the contour of the crevice. In one embodiment, plasticizers are added to formulations to give the desired flexibility. For crosslinked protein and/or non water soluble polymer formulations, possible plasticizers are glycol derivatives, phthalates, citrate derivatives, benzoates, butyl or glycol esters of fatty acids, refined mineral oils, camphor, oleic acid, castor oil, corn oil and sugar alcohols. The type and the amount of the plasticizer will control the flexibility of the composition. Preferred plasticizers for the device which comprising crosslinked protein are sorbitol and glycerin with glycerin being the most preferred plasticizer. For a device comprising a non water soluble polymer, a preferred plasticizer is triethyl citrate. The preferred amount of plasticizer is between 1, 2, 3, 4, 5, 6 or 7% and 15, 16, 17, 18, 19, 20, 21, 22, 23, 24 or 25% (w/w of the drug delivery composition), and most preferably 6-16%.

A variety of pharmacological agents may be incorporated into the precursor solutions and thus into the drug delivery devices described herein. In one embodiment, more than one pharmacological agent can be incorporated into a drug delivery device whether they be of the same therapeutic category (e.g. two or more anti-inflammatory drugs) or of different therapeutic categories, with the exception of an anti-bacterial agent (e.g. one or more anti-fungal drugs, or one or more anti-inflammatory drug and one or more anti-neoplastic drug). In one embodiment, the anti-inflammatory agent is hydrophobic or non-water soluble. The amount of drug to be incorporated into the drug delivery composition depends on the intended therapeutic use and can be determined by one skilled in the art. The drug can be present in the drug delivery composition from 0.1 to 50% (w/w), most preferably 15-45% (w/w).

A particularly preferred anti-inflammatory pharmacological agent for this delivery device is one capable of healing the periodontal tissue or one that can retard bone resorption or induce bone regrowth. Examples of such drugs are bone growth factors, bisphosphonates and flurbiprofen (FBP). Delivery devices with these drugs may be implanted surgically in the body in proximity to the site where their effect is required. The drug will be released over a prolonged period of time while the delivery device is biodegraded into harmless products. Alternately, the delivery device can be inserted into body cavities in proximity to the site of action, such as a periodontal pocket. One embodiment of this invention is to the incorporation of flurbiprofen into the delivery device and its insertion either into a periodontal pocket for the arresting of alveolar bone resorption and for the initiation of bone regrowth, or its implantation under the gum during periodontal surgery. A further preferred usage of the drug delivery device is as an adjunct treatment to periodontal surgery where it is inserted into the periodontal pockets both before and after the periodontal surgery.

Further embodiments of this invention are to the incorporation of drugs that will treat inflammation in a site in the body where the inflammation needs to be treated. Again, the drug delivery device can be inserted into body crevices that exist or are implanted in a surgical procedure. Examples of drugs whose efficacious amounts for use in the delivery device of the invention may be determined include anti-inflammatory agents including steroidal anti-inflammatory agents such as dexamethasone, budesonide, beclomethasone, and hydrocortisone.

Anti-Inflammatory agents are a well known class of pharmaceutical agents which reduce inflammation by acting on body mechanisms (Stedman's Medical Dictionary 26 ed., Williams and Wilkins, (1995); Physicians Desk Reference 51 ed., Medical Economics, (1997)).

Anti-inflammatory agents useful in the methods of the invention include Non-steroidal Anti-Inflammatory Agents (NSAIDS). NSAIDS typically inhibit the body's ability to synthesize prostaglandins. Prostaglandins are a family of hormone-like chemicals, some of which are made in response to cell injury. Specific NSAIDS approved for administration to humans include naproxen sodium, diclofenac, sulindac, oxaprozin, diflunisal, aspirin, piroxicam, indomethocin, etodolac, ibuprofen, fenoprofen, ketoprofen, mefenamic acid, nabumetone, tolmetin sodium, and ketorolac tromethamine.

Other anti-inflammatory agents useful in the methods of the invention include salicylates, such as, for example, salicilic acid, acetyl salicylic acid, choline salicylate, magnesium salicylate, sodium salicylate, olsalazine, and salsa late.

Other anti-inflammatory agents useful in the methods of the invention include cyclooxygenase (COX) inhibitors. COX catalyzes the conversion of arachidonate to prostaglandin H2 (PGH2); a COX inhibitor inhibits this reaction. COX is also known as prostaglandin H synthase, or PGH synthase. Two Cox genes, Cox-1 and Cox-2 have been isolated in several species. COX-2 is tightly regulated in most tissues and usually only induced in abnormal conditions, such as inflammation, rheumatic and osteo-arthritis, kidney disease and osteoporosis. COX-1 is believed to be constitutively expressed so as to maintain platelet and kidney function and integral homeostasis. Typical COX inhibitors useful in the methods of the invention include etodolac, celebrex, meloxicam, piroxicam, nimesulide, nabumetone, and rofecoxib.

In one embodiment of the invention, anti-inflammatory agents that can be incorporated into a polymer matrix for administration in the methods of the invention include: 3-Amino-4-hydroxybutyric Acid, Aceclofenac, Acemetacin, Acetaminosalol, Alclofenac, Alminoprofen, α-Bisabolol, Paranyline, Amfenac, Bromfenac, Benoxaprofen, Benzpiperylon, Bermoprofen, Bromosaligenin, Bucloxic Acid, Bufexamac, Bumadizon, Butibufen, Carprofen, Cinmetacin, Clidanac, Clopirac, Diclofenac, Diclofenac Sodium, Diflunisal, Ditazol, Enfenamic Acid, ε-Acetamidocaproic Acid Bendazac, Etodolac, Etofenamate, Felbinac, Fenbufen, Fenclozic Acid, Fendosal, Fenoprofen, Fentiazac, Fepradinol, Flufenamic Acid, Flunoxaprofen, Flurbiprofen, Gentisic Acid, Glucametacin, Glycol Salicylate, Ibufenac, Ibuprofen, Ibuproxam, Indomethacin, Indoprofen, Isofezolac, Isoxepac, Isoxicam, Ketoprofen, Ketorolac, Lomoxicam, Lonazola, Lonazolac, Loxoprofen, Meclofenamic Acid, Mefenamic Acid, Meloxicam, Mesalamine, Metiazinic Acid, Mofebutazone, Mofezolac, Naproxen, Niflumic Acid, Olsalazine, Oxaceprol, Oxametacine, Oxaprozin, Oxicams, Oxyphenbutazone, Paranyline, Parsalmide, Perisoxal, Phenyl Salicylate, Pirazolac, Piroxicam, Pirprofen, Pranoprofen, Proprionic Acids, Protizinic Acid, Salacetamide, Salicilic Acid, Salicylamide O-Acetic Acid, Salicylsulfuric Acid, Salsalate, Sulfasalazine, Sulindac, Suprofen, Suxibuzone, Talniflumate, Tenoxicam, Terofenamate, Tiaprofenic Acid, Tiaramide, Tinoridine, Tolfenamic Acid, Tolmetin, Tropesin, Xenbucin, Ximoprofen, Zaltoprofen, Zileuton and Zomepirac.

For any anti-inflammatory agent referred to herein by a trade name it is to be understood that either the trade name product or the active ingredient possessing anti-inflammatory activity from the product can be used.

In one embodiment, the anti-inflammatory agent and the water-insoluble polymer are present at a relative weight ratio which ranges from about 2:1 to about 1:3.

In another embodiment, the plasticizing agent and the polymer are present at a relative weight ratio which ranges from about 1:10 to about 1:2.

A further embodiment of this invention is the incorporation of the NSAID drugs listed above or morphine, codeine, or other anti pain agents for the control of pain from a localized site in the body. Implantation of the drug delivery device will allow efficacious levels of the drug to be delivered over a prolonged period at the site of action.

Further embodiments of this invention are to the incorporation of anti-neoplastic agents including methotrexate, 5-fluorouracil, tamoxifen, chlorambucil, melphalan, mercaptopurine, etoposide, and doxorubicin. Surgical implantation of the device in proximity of the tumor will give high concentration of the chemotherapeutic agent at the tumor site.

When incorporating drugs into the precursor solution it may be advantageous to include surface active agents in order to enhance solubilization of the components and to stabilize the solutions. The surface active agent may be present in amounts that vary from 0 to about 20% of the delivery device. Surfactants that may be of use in formulating the precursor solutions of this invention include polysorbate 80 (Tween 80), anionic emulsifying wax (Crodex A), and sodium lauryl sulfate. In one embodiment of this invention the surface active agents are omitted.

This precursor solution can be formed into various drug delivery devices that are polymeric solids that may be cast as films, pellets, granules, cylinders or any other convenient shape for the task at hand. The most preferable form is when cast as thin films. To form thin films the precursor mixture poured into leveled trays and is dried at room temperature. In one embodiment, the film is from about 3 to about 6 mm in length and from about 1 to about 5 mm in width and from about 0.01 to about 1.0 mm in thickness.

One preferred embodiment of the invention comprises a water soluble protein that is stable in solutions of more than 50% water/alcohol, i.e. hydrolyzed gelatin of number average molecular weight less than 20000 most preferably less than 13,000 but more than 1000. The alcohol used is ethanol and the ethanol to water ratio is between 0.1- to 1.0.

The first preferred composition of the precursor solution is hydrolyzed gelatin 6.8 parts, flurbiprofen 2.0 parts, glycerin 1.2 parts, glutaraldehyde solution (25% in water) 2.2 parts, Polysorbat 80 0.2 parts, water 72.0 parts and ethanol 15.6 parts. This formulation when dried to a thin film of 0.35 mm thickness gives a drug delivery device with the following composition:

| | |
|---|---|
| crosslinked hydrolyzed gelatin | 68.4% |
| flurbiprofen | 18.9% |
| glycerin | 10.9% |
| polysorbat 80 | 1.8% |

The second preferred composition of the precursor solution is hydrolyzed gelatin 8.1 parts, flurbiprofen 3.8 parts, glycerin 1.4 parts, glutaraldehyde solution (25% in water) 1.5 parts, Polysorbat 80 0.3 parts, water 69.0 parts and ethanol 15.9 parts. This formulation when dried to a thin film of 0.35 mm thickness gives a drug delivery device with the following composition:

| | |
|---|---|
| crosslinked hydrolyzed gelatin | 54.2% |
| flurbiprofen | 31.0% |
| glycerin | 12.9% |
| polysorbat 80 | 1.9% |

The third preferred composition of the precursor solution is hydrolyzed gelatin 11.0 parts, flurbiprofen 4.9 parts, glycerin 2.0 parts, glutaraldehyde solution (25% in water) 3.7 parts, Polysorbat 80 0.2 parts, water 59.3 parts and ethanol 19.0 parts. This formulation when dried to a thin film of 0.35 mm thickness gives a drug delivery device with the following composition:

| | |
|---|---|
| crosslinked hydrolyzed gelatin | 62.7% |
| flurbiprofen | 25.7% |
| glycerin | 10.3% |
| polysorbat 80 | 1.3% |

The thin films of the drug delivery device can be cut into any convenient shape. For use in a periodontal pocket the films can be cut to the dimensions of about 4×5×0.35 mm which is a size appropriate for inserting into a periodontal pocket. The thin film embodiments of this invention can be cut into any convenient shape for implantation in the body.

A method for the treatment of patients with periodontitis with this delivery device is another aspect of the current invention. Treatment as an adjunct to periodontal surgery, whether as an implant during surgery or as a treatment in the periodontal pocket before or after surgery or both before and after surgery should prove beneficial to the patients. An increase in bone density and bone height is expected to result from the treatment with the flurbiprofen embodiment of this invention.

Delivery devices containing steroidal or NSAID drugs can be implanted at or in proximity to a site suffering from an inflammatory process. Delivery devices containing flurbiprofen or other NSAIDs or other bone growth factors can be implanted at or in proximity to a site that requires bone growth. Delivery devices containing antibiotics, antimicrobials, or anti fungal agents can be implanted at or in proximity to a site where the action of these drugs are called for and delivery devices containing anti-neoplastic agents can be implanted at or in proximity to a tumor site.

### MiniTablets (Mini-Tab)

The main advantage of multi-particulate (MP) modified release (MR) drug delivery devices is the fact that such device may provide consistent and reliable in-vivo drug release. Mini-tab technology combines the advantages of MP dosage forms with established manufacturing techniques used in tableting. The small dimensions of Mini-tabs may contribute to such tablets being suitable for insertion into the periodontal pocket.

Technologically, the production process of Mini-Tabs is based on standard pharmaceutical tabletation equipment, thus enabling ease of preparation, versatility, flexibility, and cost effectiveness. Additional benefits of mini-tabs include excellent size uniformity, regular shape and a smooth surface, thereby offering an excellent substrate for coating with MR polymeric devices.

The formulation of such a core should contain carefully an appropriate weight ratio of active pharmaceutical ingredients (API) to inactive ingredients in order to obtain the desired release profile (either immediate release or fast release). The release of the active material can be controlled by extent of either hydrophilicity or hydrophobicity of the matrix in which the active ingredient is embedded or disperesed. Furthermore parameters such as porosity of the matrix, swelling rate and extent of the matrix, the kind and content of the disintegrant, binder, filler, glidant, hardness enhancing agent, lubricant, surface active agents, in the matrix formulation, and the coating film polymer may affect and control the release profile. Controlling the tablet properties such as disintegration, hardness, friability and etc may further affect the release profile as well.

The full contents of all publications mentioned in this specification are hereby incorporated by reference.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 shows a schematic flowchart for FBP device preparation;

FIG. 2 is a graph presenting release profiles (% accumulative release) of FBP from an FBP-device based on Example 15;

FIG. 3 is a graph showing the effect on Pocket Depth Reduction for patients treated with the following treatments: placebo chip (X); CHX chip (⋄); CHX/FBP chip (▪); and FBP chip (▴);

FIG. 4 is a graph showing the effect on bleeding on probe for patients treated with the treatments of FIG. 3; and

FIG. 5 is a graph showing the effect on clinical attachment level for patients treated with the treatments of FIG. 3.

### DETAILED DESCRIPTION OF EMBODIMENTS

### Example 1

Table 1 shows the composition of granulates prepared according to the following procedure:
Flurbiprofen (FBP) and Eudragit RS were granulated with 10% BycoM aqueous solution;
The granulates were dried.

TABLE 1
Composition of the granulates

| | Formulation No. | | |
|---|---|---|---|
| | 59-1 | 59-2 | 59-3 |
| Ingredient | Weight (g) | | |
| Flurbiprofen | 7.5 | 5.0 | 2.5 |
| Eudragit RS | 2.5 | 5.0 | 7.5 |
| *Granulation solution | 3.5 | 3.5 | 3.5 |

*10% w/w BycoM aqueous solution

### Example 2

Table 2 shows the composition of granulates prepared according to the following procedure:
FBP and Ethyl cellulose were granulated with 10% Byco M aqueous solution;
The granulates were dried.

TABLE 2
composition of the granulates

| | Formulation No | | |
|---|---|---|---|
| | 60-1 | 60-2 | 60-3 |
| Ingredient | Weight (g) | | |
| Flurbiprofen | 7.5 | 5.0 | 2.5 |
| Ethyl cellulose | 2.5 | 5.0 | 7.5 |
| *Granulation solution | 3.5 | 3.5 | 3.5 |

*10% w/w Byco M in aqueous solution

### Example 3

Table 3 shows the composition of granulates prepared according to the following procedure:
FBP, Ethyl cellulose and Byco M were granulated with ethanol;
The granulates were dried.

TABLE 3
composition of the granulates

| | Formulation No. | | |
|---|---|---|---|
| | 76-1 | 76-2 | 76-3 |
| Ingredient | Weight (g) | | |
| Flurbiprofen | 4.0 | 4.0 | 4.0 |
| Ethyl cellulose | 4.0 | 4.0 | 6.0 |
| Byco M | 2.0 | 4.0 | — |
| *Granulation solution | 5.0 | 5.0 | 5.0 |

*Ethanol

### Example 4

Table 4 shows the composition of granulates prepared according to the following procedure:
FBP, Ethyl cellulose and Pullulane were granulated with ethanol;
The granulates were dried.

TABLE 4
composition of the granulates

| | Formulation No. | | |
|---|---|---|---|
| | 78-1 | 78-2 | 78-3 |
| Ingredient | Weight (g) | | |
| Flurbiprofen | 3.0 | 3.0 | 3.0 |
| Ethyl cellulose | 1.0 | 0.5 | 1.5 |
| Pullulane | 1.0 | 1.5 | 0.5 |
| *Granulation solution | 2.5 | 2.5 | 2.5 |

*Ethanol

### Example 5

Table 5 shows the composition of granulates prepared according to the following procedure:
FBP and Ethyl cellulose were granulated with 30% aquacoat solution;
The granulates were dried.

TABLE 5
composition of the granulates

Formulation No.

| | | | |
|---|---|---|---|
| 80-1 | 80-2 | 80-3 | 80-4 |

| | |
|---|---|
| Ingredient | Weight (g) |

| | | | | |
|---|---|---|---|---|
| Flurbiprofen | 100 | 5.1 | 7.5 | 2.5 |
| Ethyl cellulose | — | 5.1 | 2.5 | 7.5 |
| *Granulation solution | 3.0 | 3.3 | 3.2 | 3.7 |

*30% w/w Aquacoat

### Example 6

Table 6 shows the composition of granulates prepared according to the following procedure:
FBP and Byco M were granulated with 30% Kollicoat solution;
The granulates were dried.

TABLE 6
composition of the granulates

| | Formulation No. | | |
|---|---|---|---|
| | 81-1 | 81-2 | 81-3 |
| Ingredient | Weight (g) | | |
| Flurbiprofen | 6.0 | 4.0 | 2.0 |
| Byco M | 2.0 | 4.0 | 6.0 |
| *Granulation solution | 3.0 | 3.0 | 3.0 |

*30% Kollicoat

### Example 7

Table 7 shows the composition of granulates prepared according to the following procedure:
FBP and ethyl cellulose were granulated with 30% Kollicoat solution.
The granulates were dried.

TABLE 7
composition of the granulates

| | Formulation No. | | |
|---|---|---|---|
| | 82-1 | 82-2 | 82-3 |
| Ingredient | Weight (g) | | |
| Flurbiprofen | 6.0 | 4.0 | 2.0 |
| Ethyl cellulose | 2.0 | 4.0 | 6.0 |
| *Granulation solution | 3.0 | 3.0 | 3.0 |

*30% Kollicoat

### Example 8

Table 8 shows the composition of granulates prepared according to the following procedure:
FBP, ethyl cellulose and Eudragit were granulated together with 30% Kollicoat solution;
The granulates were dried.

TABLE 8
composition of the granulates

| | Formulation No. | | |
|---|---|---|---|
| | 83-1 | 83-2 | 83-3 |
| Ingredient | Weight (g) | | |
| Flurbiprofen | 4.0 | 4.0 | 2.0 |
| Ethyl cellulose | 4.0 | 2.0 | — |
| Eudragit L-100 | — | 2.0 | 4.0 |
| *Granulation solution | 3.5 | 3.5 | 3.5 |

*10% w/w of crosslinking solution of Byco M with glutaraldehyde (15% on polymer)

### Example 9

The liquid precursor composition was prepared by the two following steps:
Granulation
Film solution

### The Granulation Step:

Table 9-1 shows the composition of granulates prepared according to the following procedure:
FBP, ethyl cellulose, Eudragit L-100 and hydrolyzed fish gelatin. (Byco M) were granulated with 14% Byco M in aqueous solution;
The granulates were dried.

TABLE 9-1
FBP granulated compositions

| | Formulation No | | | | |
|---|---|---|---|---|---|
| | 609-65 | 609-66 | 609-68 | 609-69 | 609-70 |
| Ingredient | Weight (g) | | | | |
| Flurbiprofen | 4.0 | 4.0 | 4.0 | 4.0 | 4.0 |
| Ethyl Cellulose | 2.2 | 3.3 | 1.1 | — | 4.4 |
| Eudragit L-100 | 2.2 | 1.1 | 3.3 | 4.4 | — |
| Byco M | 0.7 | 0.7 | 0.7 | 0.7 | 0.7 |
| Purified water | 5.0 | 5.0 | 5.0 | 5.0 | 5.0 |

### The Film Solution Step:

The compositions summarized in Table 9-2 were prepared according to the following procedure:
The granulates were dispersed in ethanol as a solvent and the resulting dispersion was mixed for 30 min;
Triethyl citrate (TEC) was added and the mixing was continued for additional 10 min;
Polysorbate was added and mixing of the resulting mixture was continued for further 10 min.

TABLE 9-2
Film solution compositions

| | Formulation No. | | | | |
|---|---|---|---|---|---|
| | 609-65 | 609-66 | 609-68 | 609-69 | 609-70 |
| Ingredient | Weight (g) | | | | |
| FBP Granulate | 8.7 | 8.7 | 8.7 | 8.7 | 8.7 |
| Triethyl citrate | 0.6 | 0.6 | 0.6 | 0.6 | 0.6 |
| Polysorbat 80 | 0.3 | 0.3 | 0.3 | 0.3 | 0.3 |
| Ethanol | 30.8 | 30.8 | 30.8 | 30.8 | 30.8 |

### Example 10

The liquid precursor composition was prepared by the two following steps:
Granulation
Film solution

### Granulation Step:

Table 10-1 shows the composition of the granulates prepared according to the following procedure:
FBP and ethyl cellulose were granulated with 10% w/w of Byco M (hydrolyzed fish gelatin) crosslinking with glutaraldehyde (15% on polymer) in an aqueous solution;
The granulates were dried.

TABLE 10-1
FBP granulate compositions

| | Formulation No | | | | |
|---|---|---|---|---|---|
| | 84-1 | 84-2 | 84-3 | 84-4 | 84-5 |
| Ingredient | Weight (g) | | | | |
| Flurbiprofen | 1.5 | 3.0 | 5.0 | 7.0 | 8.5 |
| Ethyl Cellulose | 8.5 | 7.0 | 5.0 | 3.0 | 1.5 |
| *Granulation solution | 3.5 | 3.5 | 3.5 | 3.5 | 3.5 |

*10% w/w of crosslinking solution of Byco M with glutaraldehyde (15% on polymer)

### The Film Solution Step:

The compositions summarized in Table 10-2 were prepared according to the following procedure:
The granulate was dispersed in ethanol as a solvent and the dispersion was mixed for 30 min;
triethyl citrate (TEC) was added and mixing of the resulting mixture was continued for further 15 min.

TABLE 10-2
Film solution compositions

| | Formulation No. | | | | |
|---|---|---|---|---|---|
| | 84-1 | 84-2 | 84-3 | 84-4 | 84-5 |
| Ingredient | Weight (g) | | | | |
| FBP Granulate | 8.0 | 8.0 | 8.0 | 8.0 | 8.0 |
| Triethyl citrate | 0.4 | 0.4 | 0.4 | 0.4 | 0.4 |
| Ethanol | 40.0 | 40.0 | 40.0 | 40.0 | 40.0 |
| Total | 48.4 | 48.4 | 48.4 | 48.4 | 48.4 |

### Example 11

The liquid precursor composition was prepared by the following steps:
Granulation
Film solution

### Granulation Step:

Table 11-1 shows the composition of the granulates according to the following procedure:
FBP and Eudragit L-100 was granulated with 10% w/w of Byco M (hydrolyzed fish gelatin) crosslinking with glutaraldehyde (15% on polymer) in an aqueous solution
The garnuletes were dried.

TABLE 11-1
FBP granulate compositions

| | Formulation No | | | | |
|---|---|---|---|---|---|
| | 85-1 | 85-2 | 85-3 | 85-4 | 85-5 |
| Ingredient | Weight (g) | | | | |
| Flurbiprofen | 1.5 | 3.0 | 5.0 | 7.0 | 8.5 |
| Eudragit 1-100 | 8.5 | 7.0 | 5.0 | 3.0 | 1.5 |
| *Granulation | 3.5 | 3.5 | 3.5 | 3.5 | 3.5 |
| solution | | | | | |

*10% w/w of crosslinking solution of Byco M with glutaraldehyde (15% on polymer)

### The Film Solution Step:

The compositions summarized in Table 11-2 were prepared according to the following procedure:
The granulate was dispersed in ethanol as a solvent and the dispersion was mixed for 30 min;
triethyl citrate (TEC) was added and mixing of the resulting mixture was continued for further 15 min.

TABLE 11-2
Film solution compositions

| | Formulation No. | | | | |
|---|---|---|---|---|---|
| | 85-1 | 85-2 | 85-3 | 85-4 | 85-5 |
| Ingredient | Weight (g) | | | | |
| FBP Granulate | 8.0 | 8.0 | 8.0 | 8.0 | 8.0 |
| Triethyl citrate | 0.4 | 0.4 | 0.4 | 0.4 | 0.4 |
| Ethanol | 40.0 | 40.0 | 40.0 | 40.0 | 40.0 |
| Total | 48.4 | 48.4 | 48.4 | 48.4 | 48.4 |

### Example 12

The liquid precursor compositions in Table 12 were prepared according to the following procedure:
1. Eudragit L-100 was dissolved in ethanol as a solvent and the solution was mixed for 15 min.
2. Triethyl citrate (TEC) was added and the mixing was continued for additional 15 minutes.
3. Ethyl cellulose was added and the solution was mixed for additional 15 minutes.
4. FBP in an alcoholic solubilizer mixture was dissolved and then added to the above solution and the mixture was mixed for 15 min.
5. Byco M aqueous solution was then added and mixing of the resulting mixture was continued for further 15 min.

TABLE 12
liquid precursor compositions

Formulation

| | | | |
|---|---|---|---|
| 609-61 | 609-62 | 609-63 | 609-64 |

| | |
|---|---|
| Ingredient | Weight (g) |

| | | | | |
|---|---|---|---|---|
| Flurbiprofen | 2.7 | 2.7 | 2.7 | 3.8 |
| Eudragit L-100 | 2.8 | 0.3 | 0.8 | 2.4 |
| Ethyl cellulose | 0.3 | 2.8 | 4.0 | 2.4 |
| Triethyl citrate | 0.7 | 0.7 | 0.7 | 0.6 |
| Byco M | 0.4 | 0.4 | 0.4 | 0.5 |
| Polysorbat 80 | 0.2 | 0.2 | 0.2 | 0.3 |
| Ethanol | 29.3 | 29.3 | 43.2 | 47.2 |
| Purified water | 2.4 | 2.4 | 2.4 | 2.4 |
| Total | 38.8 | 38.8 | 54.4 | 59.6 |

### Example 13

The liquid precursor compositions in Table 13 were prepared according to the following procedure:
1. Eudragit L-100 was dissolved in ethanol as a solvent and the solution was mixed for 15 min.
2. Ethyl cellulose was added and the solution was mixed for 15 min
3. FBP was added and mixed for additional 15 min
4. Polysorbate 80 was added and mixing was continued for 15 min
5. triethyl citrate (TEC) was added and mixed for additional 15 min
6. Byco M aqueous solution was added and mixed for further 15 min.

TABLE 13
liquid precursor compositions

Formulation

| | | | |
|---|---|---|---|
| 609-67 | 609-71 | 609-72 | *609-74 |

| | |
|---|---|
| Ingredient | Weight (g) |

| | | | | |
|---|---|---|---|---|
| Flurbiprofen | 4.0 | 4.0 | 4.0 | 4.3 |
| Eudragit L-100 | 2.2 | 1.1 | 3.3 | — |
| Ethyl cellulose | 2.2 | 3.3 | 1.1 | 4.7 |
| Triethyl citrate | 0.6 | 0.6 | 0.6 | — |
| Byco M | 0.7 | 0.7 | 0.7 | 0.7 |
| Polysorbat 80 | 0.3 | 0.3 | 0.3 | 0.3 |
| Ethanol | 33.5 | 30.8 | 30.8 | 30.8 |
| Purified water | 5.0 | 5.0 | 5.0 | 5.0 |
| Total | 48.5 | 45.8 | 45.8 | 45.8 |

*The formulation without Eudragit L-100 and Triethyl citrate.

### Example 14

The liquid precursor compositions (Table 14) were prepared according to the following procedure:
1. Hydrolyzed gelatin was dissolved in a mixture of water/alcohol and the solution was mixed for 45 min. Glycerin was added and mixed for 15 min.
2. Glutaraldehyde solution 10% was added and the solution was mixed for additional 15 min
3. FBP in an alcoholic solubilizer mixture was added and the resulting mixture was mixed for further 15 min.

TABLE 14
liquid precursor compositions

Formulation

| | 16-1 | 16-2 | 16-3 | 16-4 | 16-5 |
|---|---|---|---|---|---|
| Ingredient | Weight (g) | | | | |
| FBP | 48 | 40 | 48 | 48 | 40 |
| BYCO M 1 | 80 | 85 | 80 | 78 | 90 |
| Glycerin | 12 | 15 | 20 | 20 | 16 |
| GA (25%) | 16 | 16 | 16 | 24 | 30 |
| Polysorbate 80 | 3 | 3 | 3 | 3 | 2 |
| Ethanol | 195 | 167 | — | — | 156 |
| Purified water | 346 | 724 | 864 | 864 | 486 |
| TOTAL | 700 | 1050 | 1031 | 1037 | 820 |

### Example 15

### Scaled Up Production Process

The production process was carried out as follows:
1. Hydrolyzed gelatin was dissolved in an aqueous/alcohol mixture and the solution was mixed for 45 min. Glycerin was added and the mixing was continued for additional 15 min
2. Glutaraldehyde solution 10% was added and the solution was mixed for additional 15 min
3. FBP in an alcoholic solubilizer mixture was added and the resulting mixture was mixed for further 15 min.
4. The formulation of the precursor is shown in Table 15

TABLE 15
liquid precursor compositions

| Formulation | W (gr) | W (%) |
|---|---|---|
| BYCO M¹ | 390 | 6.8 |
| Glycerin | 67 | 1.2 |
| GA (25%)² | 123 | 2.2 |
| Polysorbate 80 | 11 | 0.2 |
| FBP³ | 116 | 2.0 |
| Ethanol | 892 | 15.6 |
| P.W⁴ | 4121 | 72.0 |
| TOTAL | 5720 | 100.0 |

¹BYCO M - hydrolyzed gelatin
²GA (25%) - 25% aqueous solution of glutaraldehyde
³FBP—Flurbiprofen
⁴P.W.—purified water

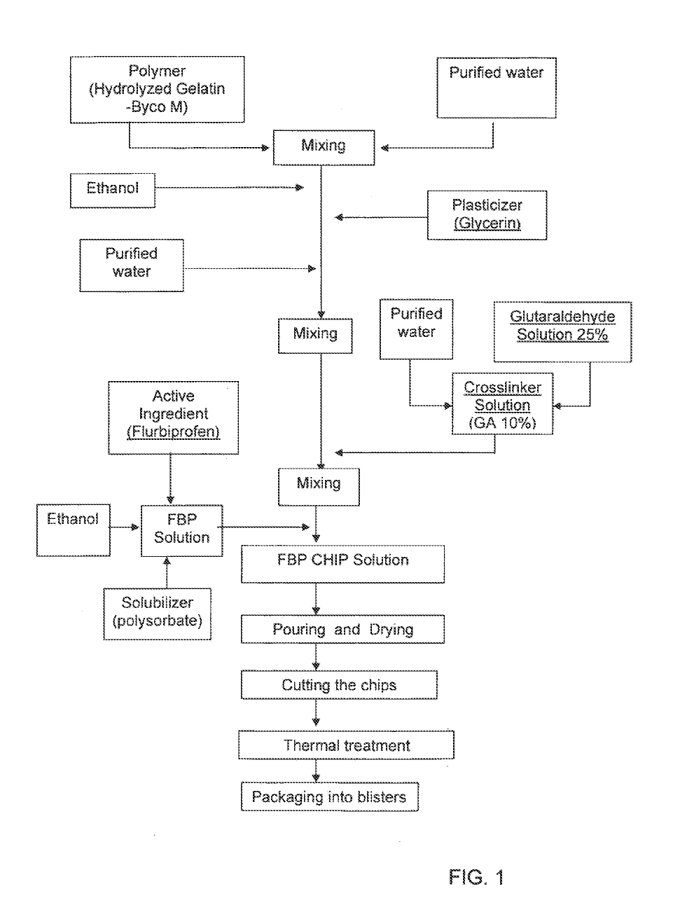

The preparation process of the FBP-device is shown in FIG. 1.

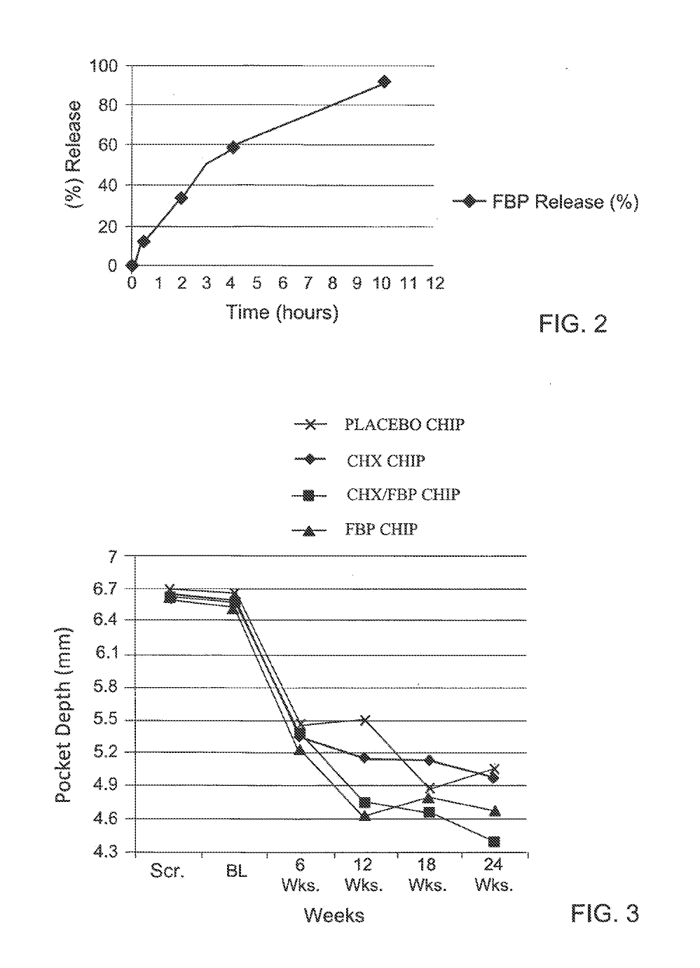

The in vitro release profile of FBP from the FBP-device is shown in FIG. 2

### Example 16

### Release Profiles from FBP Granulates

The accumulative release of FBP from different granules (see Examples 1-11) was determined using a dissolution method. The dissolution test was performed in a 900 ml solution at 37° C. Phosphate buffer pH-4.5 was used as the medium of the dissolution. The paddle speed was set at 100 rpm. The amount of FBP released from the granulates at each point of time was quantified automatically by U.V method. The results are summarized in Tables 16-1 and 16-2.

TABLE 16-1
The release of FBP from the FBP granulates

| | Batch# | | | | | | | | | | | |
|---|---|---|---|---|---|---|---|---|---|---|---|---|
| | 59-1 | 59-2 | 59-3 | 60-1 | 60-2 | 60-3 | 78-1 | 78-2 | 78-3 | 83-1 | 83-2 | 83-3 |
| Time (hr) | % Release | | | | | | | | | | | |
| 0 | 0 | 0 | 0 | 0 | 0 | 0 | 0 | 0 | 0 | 0 | 0 | 0 |
| 0.5 | 12 | 12 | 20 | 47 | 33 | 19 | 5 | 0 | 0 | 4 | 9 | 7 |
| 1 | 21 | 21 | 35 | 70 | 60 | 9 | 10 | 0 | 3 | 11 | 22 | 17 |
| 2 | 34 | 33 | 53 | 83 | 79 | 9 | 17 | 0 | 7 | 23 | 38 | 29 |
| 3 | 45 | 42 | 64 | 89 | 88 | 1 | 23 | 0 | 9 | 33 | 51 | 37 |
| 4 | 56 | 50 | 72 | 93 | 92 | 8 | 28 | 4 | 12 | 41 | 59 | 44 |
| 6 | 82 | 71 | 85 | 96 | 95 | 3 | 34 | 12 | 18 | 50 | 67 | 50 |
| 8 | 88 | 77 | 88 | 98 | 97 | 6 | 39 | 19 | 23 | 57 | 73 | 56 |
| 10 | 90 | 82 | 88 | 98 | 98 | 7 | 43 | 22 | 26 | 62 | 77 | 60 |

TABLE 16-2
The release of FBP from the FBP granulates

| | Batch# | | | | | | | | | |
|---|---|---|---|---|---|---|---|---|---|---|
| | 84-1 | 84-2 | 84-3 | 84-4 | 84-5 | 85-1 | 85-2 | 85-3 | 85-4 | 85-5 |
| Time (hr) | % Release | | | | | | | | | |
| 0 | 0 | 0 | 0 | 0 | 0 | 0 | 0 | 0 | 0 | 0 |
| 0.5 | 0 | 0 | 12 | 61 | 71 | 19 | 15 | 9 | 31 | 23 |
| 1 | 2 | 1 | 2 | 74 | 81 | 35 | 29 | 19 | 50 | 43 |
| 2 | 6 | 9 | 32 | 80 | 84 | 50 | 46 | 30 | 66 | 60 |
| 3 | 10 | 17 | 40 | 83 | 86 | 62 | 56 | 40 | 73 | 72 |
| 4 | 13 | 24 | 45 | 85 | 86 | 69 | 65 | 47 | 78 | 79 |
| 6 | 17 | 31 | 51 | 86 | 87 | 76 | 74 | 55 | 83 | 84 |
| 8 | 19 | 38 | 56 | 87 | 87 | 80 | 79 | 61 | 87 | 88 |
| 10 | 22 | 42 | 59 | 88 | 88 | 82 | 83 | 65 | 89 | 90 |

### Example 17

### Release Profiles from FBP Granulates

The accumulative release of FBP from different granules was determined using a dissolution method. The dissolution test was performed in a 900 ml at 37° C. Buffer phosphate pH-6.8 was used as the medium of the dissolution. The paddle speed was set at 100 rpm. The amount of FBP released from the granulates at each point of time was quantified automatically by U.V method. The results are summarized in Table 17.

TABLE 17
The release of flurbiprofen from the FBP granulates

| | Batch# | | | | | | |
|---|---|---|---|---|---|---|---|
| | 76-1 | 76-2 | 76-3 | 82-1 | 82-2 | 84-1 | 84-2 |
| Time (hr) | % Release | | | | | | |
| 0 | 0 | 0 | 0 | 0 | 0 | 0 | 0 |
| 0.5 | 63 | 70 | 4 | 93 | 88 | 47 | 67 |
| 1 | 67 | 79 | 6 | 93 | 88 | 62 | 91 |
| 2 | 70 | 82 | 8 | 93 | 89 | 65 | 91 |
| 3 | 71 | 84 | 11 | 94 | 90 | 68 | 92 |
| 4 | 71 | 85 | 13 | 94 | 90 | 69 | 93 |
| 6 | 72 | 86 | 16 | 94 | 90 | 71 | 95 |
| 8 | 73 | 88 | 18 | 95 | 90 | 72 | 96 |
| 10 | 75 | 89 | 21 | 95 | 91 | 75 | 97 |

### Example 18

### Release profiles from FBP-device (FBP Chip) prepared from granulates

The accumulative release of FBP from different FBP-devices was determined using a dissolution method. The dissolution test was performed in a 900 ml solution at 37° C. Buffer phosphate pH-6.8 was used as the medium of the dissolution. The basket speed was set at 100 rpm. 5 chips were placed in each vessel. The amount of FBP released from the devices at each point of time was quantified automatically by U.V method. The results are summarized in Table 18.

TABLE 18
The release of FBP from the FBP-device (FBP Chip)
prepared from granulates

| | Batch# | | | | | | |
|---|---|---|---|---|---|---|---|
| | 609-65 | 609-66 | 609-68 | 609-70 | 84-1 | 84-2 | 84-3 |
| Time (hr) | % Release | | | | | | |
| 0 | 0 | 0 | 0 | 0 | 0 | 0 | 0 |
| 0.5 | 21 | 9 | 15 | 4 | 1 | 2 | 4 |
| 1 | 33 | 15 | 28 | 6 | 1 | 2 | 5 |
| 2 | 49 | 23 | 52 | 8 | 1 | 3 | 7 |
| 3 | 59 | 29 | 74 | 11 | 3 | 3 | 8 |
| 4 | 67 | 34 | 90 | 13 | 3 | 4 | 9 |
| 6 | 81 | 44 | 100 | 16 | 3 | 5 | 11 |
| 8 | 91 | 53 | 102 | 18 | 4 | 6 | 13 |
| 10 | 98 | 60 | 103 | 21 | 4 | 7 | 14 |

### Example 19

### Release Profiles from FBP-Device (FBP Chip) Prepared from Precursor

The accumulative release of FBP from different FBP-devices prepared from precursor was determined using a dissolution method. The dissolution test was performed in a 900 ml solution at 37° C. Buffer phosphate pH-6.8 was used as the medium of the dissolution. The basket speed was set at 100 rpm. 5 chips were placed in each vessel. The amount of FBP released from the devices at each point of time was quantified automatically by U.V method. The results are summarized in Table 19.

TABLE 19
The release of FBP from the FBP-device
prepared from precursor

| | Batch# | | | | | | |
|---|---|---|---|---|---|---|---|
| Time | 609-61 | 609-62 | 609-63 | 609-64 | 609-67 | 609-72 | 609-74 |
| (hr) | % Release | | | | | | |
| 0 | 0 | 0 | 0 | 0 | 0 | 0 | 0 |
| 0.5 | 23 | 32 | 7 | 25 | 10 | 20 | 4 |
| 1 | 43 | 41 | 11 | 48 | 18 | 34 | 5 |
| 2 | 93 | 51 | 15 | 73 | 32 | 53 | 7 |
| 3 | 100 | 57 | 18 | 82 | 44 | 67 | 9 |
| 4 | 102 | 62 | 21 | 87 | 55 | 79 | 11 |
| 6 | 102 | 69 | 25 | 93 | 74 | 97 | 14 |
| 8 | 102 | 74 | 30 | 95 | 85 | 100 | 16 |
| 10 | 102 | 79 | 33 | 97 | 92 | 100 | 18 |

### Example 20

### Clinical Study

### Study Objective:

To determine the effect of the placement in a periodontal pocket of each of the following chips on probing pocket depth (PPD):
PerioChip Plus (flurbiprofen/chlorhexidine—FBP/CHX) formulation
PerioChip (chlorhexidine) formulation
Flurbiprofen Chip formulation
Placebo Chip formulation
   Study Duration: 25 weeks.
   Study Treatments Four treatment arms.

Treatment 1—PerioChip Plus (flurbiprofen/chlorhexidine—FBP/CHX) formulation

Treatment 2—PerioChip (chlorhexidine—CHX) formulation

Treatment 3—Flurbiprofen Chip formulation

Treatment 4—Placebo Chip formulation

Dosage: The dosage for the first treatment arm consists of a single PerioChip Plus (flurbiprofen/chlorhexidine—FBP/CHX) formulation, containing 1.5 mg flurbiprofen and 2.5 mg chlorhexidine.

The dosage for the second treatment arm consists of a single PerioChip (chlorhexidine—CHX) formulation, containing 2.5 mg chlorhexidine,

The dosage for the third treatment arm consists of a single Flurbiprofen formulation, containing 1.5 mg flurbiprofen.

The forth arm consists of a placebo Chip formulation.

### Subjects:

Inclusion Criteria:
Signed informed consent form
Good general health
Male or female subjects aged >25 years old
Minimum of 8 natural teeth
Availability for the 25 weeks duration of the study
Periodontal disease on a natural teeth characterized by the presence of at least 2 teeth with periodontal pockets of 6-9 mm in depth (potential target teeth) in order to reach baseline (day 1) with periodontal pockets of 5-8 mm in depth, without involving the apex of the tooth.
Females of childbearing potential must be non pregnant at entry and agree to use an adequate method of birth control during the study.

### End-Point Criteria:

At 24 weeks, relative to baseline, the mean reductions in probing pocket depth (PPD) is used as primary efficacy endpoint. Additional primary endpoints are clinical attachment levels (CAL) and bleeding on probing (BOP) in the target pockets selected at baseline, measured at weeks 24.

PPD measurements at 6, 12 and 18 weeks are used as secondary endpoints. Additional secondary endpoints are clinical attachment levels (CAL) and bleeding on probing (BOP) in the target pockets selected at baseline, measured at weeks 6, 12 and 18.

### Study Rationale

Clinical studies using devices and topical administrations of FBP demonstrated that FBP reduces gingival inflammation, prevents the progression of alveolar bone loss in subjects with periodontal disease and, in some cases, causes bone mass gain. Dexcel Pharma Technologies Ltd. developed a drug delivery device, the PerioChip® (chlorhexidine gluconate 2.5 mg), based on local application. This device consists of a biodegradable polymer of cross linked hydrolyzed gelatin, which releases chlorhexidine gluconate directly into the periodontal pocket over a period of about seven days. The FBP/CHX chip drug delivery device is similar to the PerioChip®, with the addition of a second active ingredient flurbiprofen 1.5 mg. It is anticipated that treatment with the FBP/CHX chip will be effective since the active ingredient will be released directly into the pocket, with concentrations of drug maintained over a sustained period.

The local use of the FBP/CHX chip at the inflammatory pocket (site) would avoid potential NSAID-related adverse events in the GI tract and other body devices.

The slow release of FBP would provide long-term maintenance of therapeutic levels of the drug without concerns for subject compliance.

### Objective

The objective of this clinical study is to determine the efficacy and safety of the placement of a FBP/CHX chip containing a combination of flurbiprofen 1.5 mg and chlorhexidine gluconate 2.5 mg on probing pocket depth, clinical attachment level (CAL), and bleeding on probing. These results are compared to those for CHX chip (second arm), FBP chip (third arm) and Placebo chip (fourth arm).

These treatments were applied to 2 teeth with periodontal pockets of 5-8 mm in depth (target teeth), without involving the apex of the tooth. At 24 weeks, relative to baseline, the mean reductions in probing pocket depth (PPD), the clinical attachment level (CAL) and the bleeding on probing (BOP) in the target pockets (sites) selected at baseline are used as primary efficacy endpoint. PPD measurements.

6, 12 and 18 weeks were used as secondary endpoints, as well as clinical attachment Level (CAL), and bleeding on probing (BOP) at 6, 12 and 18 weeks.

The duration of subject follow up was 24 weeks, with interim visits at 6, 12 and 18 weeks.

### Study Subjects

Number of Subjects:

Eighty (80) male and female subjects: 25 subjects in the PerioChip Plus arm;

25 subjects in the PerioChip arm; 15 subjects in the Flurbiprofen Chip arm;

and 15 subjects in the Placebo Chip arm, with moderate to advanced adult periodontitis were entered into the study following written informed consent. To be eligible for this study subjects must have, at screening, at least 2 potential target pockets with a PPD of 6-9 mm in order to reach baseline (day 1) with periodontal pockets of 5-8 mm in depth, without involving the apex of the tooth.

### Exclusion Criteria

Presence of more than 2 adjacent periodontal pockets on the same potential target tooth.
Periodontal pockets of more than 9 mm in depth, and with class 2 or 3 furcation involvement, on the potential target teeth.

### Plan of Study

Two (2) target pockets in each subject were used for chip placement in the study. Those pockets in each subject meeting the entrance criteria were treated in one of the ways described below. The specific treatment of each target pocket was initiated at day 1 (baseline).

Treatment 1: (FBP/CHX chip) FBP/CHX chip, consisting of 2.5 mg chlorhexidine gluconate and 1.5 mg flurbiprofen formulated in a biodegradable cross linked gelatin matrix was placed in each one of the target pockets (PPD of 5-8 mm), one in each tooth, for a total of 2 treated pockets in each subject mouth.

Treatment 2: (CHX chip) CHX chip, consisting of 2.5 mg chlorhexidine gluconate formulated in a biodegradable cross linked gelatin matrix was placed in each one of the target pockets (PPD of 5-8 mm), one in each tooth, for a total of 2 treated pockets in each subject mouth.

Treatment 3: (FBP chip) FBP chip, consisting of 1.5 mg flurbiprofen formulated in a biodegradable cross linked gelatin matrix was placed in each one of the target pockets (PPD of 5-8 mm), one in each tooth, for a total of 2 treated pockets in each subject mouth.

Treatment 4: (Placebo chip)

Placebo chip (not consisting of active treatment) formulated in a biodegradable crosslinked gelatin matrix was placed in each one of the target pockets (PPD of 5-8 mm), one in each tooth, for a total of 2 treated pockets in each subject mouth.

### Efficacy Analysis

### Probing Pocket Depth (PPD)

PPD is the measurement of the distance from the coronal edge of the gingival margin to the base of the pocket. PPD was measured at four sites per tooth: mesio-buccal, mid-buccal, disto-buccal, mid-lingual. Measurement was taken with a standard 15-mm University of North Carolina (UNC) periodontal probe. For recording pocket depth, the probe tip is placed at the bottom of the pocket and the pocket depth read directly from the millimetres markings on the probe.

### Recession (R)

Recession is defined as the distance in millimetres that the free gingival margin has migrated apically from the cemento-enamel junction (CEJ) at the same site that PPD was measured using a standard 15-mm University of North Carolina (UNC) periodontal probe.

### Clinical Attachment Level (CAL)

Loss of attachment is defined as the distance in millimetres that the base of the pocket has migrated apically from the CEJ. CAL was calculated at the same site mentioned above, by adding the recession measurement (R) to the PPD measurement.

### Bleeding on Probing (BOP)

BOP was measured at the same site immediately after measuring the PPD. The scoring device used for recording the BOP is a dichotomous one:

0=No bleeding
1=Bleeding on probing to the base of the pocket

The primary efficacy parameter was the change from baseline for the mean reduction in PPD for the treated pockets.

Additional primary efficacy parameters were:

Proportion of pockets with at least 1 mm reduction in PPD.

Improvement in CAL relative to baseline for the treated pockets.

Reduction of BOP scores relative to baseline. BOP score of 0 (=no bleeding), or 1 (=bleeding) was assigned to each target pocket at baseline and post-baseline.

The primary time-point for all analyses was at 24 weeks. PPD measurements for the treated pockets at 6, 12 and 18 weeks was used as secondary endpoints. Additional secondary endpoints were CAL and BOP in the target pockets selected at baseline, measured at weeks 6, 12 and 18.

### Results

### I. Pocket Depth

Intent to Treat Patients (ITT) Pocket Depth (PD) Reduction Results:

| Study Visits | Scr. | BL | 6 Wks. | 12 Wks. | 18 Wks. | 24 Wks. |
|---|---|---|---|---|---|---|
| | Treatment | | | | | |
| | CHX CHIP | | | | | |
| Mean | 6.64 | 6.6 | 5.36 | 5.16 | 5.14 | 4.98 |
| STD | 1.01 | 0.83 | 1.31 | 1.1 | 1.15 | 1.28 |
| SE | 0.14 | 0.12 | 0.18 | 0.17 | 0.17 | 0.2 |
| Δ mm from BL | | | −1.24 | −1.44 | −1.46 | −1.62 |
| Δ % from BL | | | −18.79 | −21.83 | −22.18 | −24.6 |
| | Treatment | | | | | |
| | CHX/FBP CHIP | | | | | |
| Mean | 6.62 | 6.58 | 5.39 | 4.75 | 4.66 | 4.39 |
| STD | 1.03 | 0.99 | 1.33 | 1.24 | 1.36 | 1.24 |
| SE | 0.15 | 0.14 | 0.2 | 0.19 | 0.22 | 0.2 |
| Δ mm from BL | | | −1.19 | −1.83 | −1.92 | −2.19 |
| Δ % from BL | | | −18.14 | −27.81 | −29.21 | −33.21 |
| | Treatment | | | | | |
| | FBP CHIP | | | | | |
| Mean | 6.63 | 6.53 | 5.23 | 4.65 | 4.79 | 4.67 |
| STD | 0.96 | 0.82 | 1.07 | 1.26 | 1.44 | 1.63 |
| SE | 0.18 | 0.15 | 0.20 | 0.25 | 0.29 | 0.33 |
| Δ mm from BL | | | −1.3 | −1.88 | −1.74 | −1.87 |
| Δ % from BL | | | −19.9 | −28.77 | −26.66 | −28.57 |
| | Treatment | | | | | |
| | PLACEBO CHIP | | | | | |
| Mean | 6.69 | 6.67 | 5.46 | 5.5 | 4.88 | 5.05 |
| STD | 1.07 | 1.03 | 0.78 | 1.32 | 1.39 | 1.4 |
| SE | 0.2 | 0.19 | 0.16 | 0.27 | 0.28 | 0.3 |
| Δ mm from BL | | | −1.21 | −1.17 | −1.79 | −1.62 |
| Δ % from BL | | | −18.13 | −17.5 | −26.88 | −24.32 |

The results are also summarized in FIG. 3.

Bleeding on Probe (BOP) Results:

| | BOP | | | | |
|---|---|---|---|---|---|
| Treatment | BL | 6 Wks. | 12 Wks. | 18 Wks. | 24 Wks. |
| CHX CHIP | 0.94 | 0.66 | 0.78 | 0.66 | 0.64 |
| CHX/FBP CHIP | 0.94 | 0.59 | 0.59 | 0.50 | 0.61 |
| FBP CHIP | 0.91 | 0.73 | 0.54 | 0.63 | 0.58 |
| PLCEBO CHIP | 1.00 | 0.75 | 0.71 | 0.75 | 0.73 |

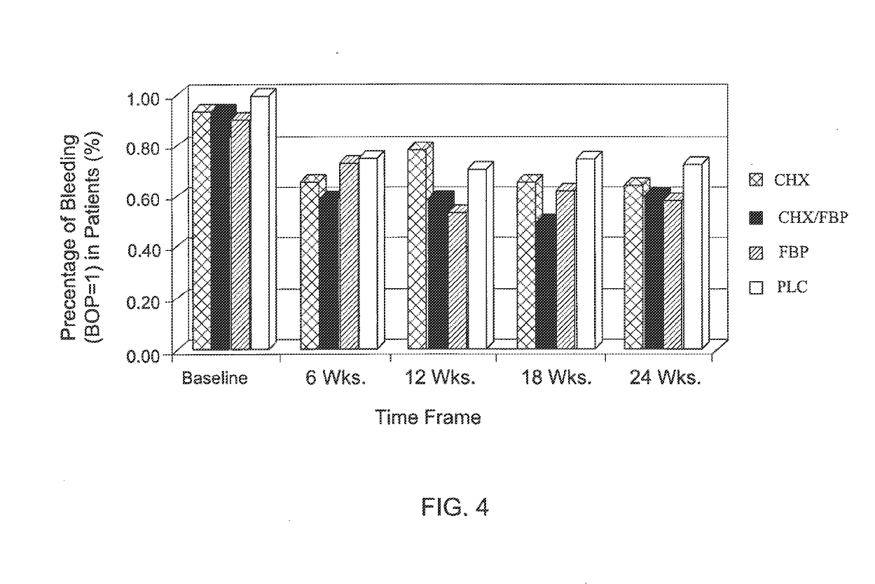

The results are also summarized in FIG. 4.

### Clinical Attachment Level (CAL) Results

| | CAL | | | | |
|---|---|---|---|---|---|
| Treatment | BL | 6 Wks. | 12 Wks. | 18 Wks. | 24 Wks. |
| CHX CHIP | 7.64 | 6.56 | 6.33 | 6.70 | 6.60 |
| CHX/FBP CHIP | 7.40 | 6.64 | 5.84 | 5.89 | 5.66 |
| FBP CHIP | 7.34 | 6.33 | 5.85 | 6.04 | 6.17 |
| PLCEBO CHIP | 7.67 | 7.04 | 7.04 | 6.67 | 6.73 |

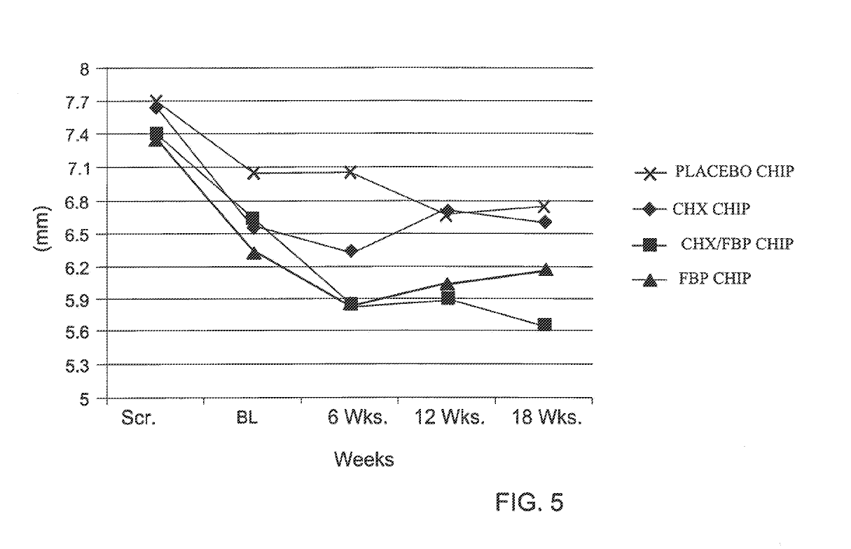

The results are also summarized in FIG. 5.

Surprisingly, the FBP chip gave similar results to the CHX/FBP chip. Thus, a chip containing an anti-inflammatory agent alone may be used to obtain the same therapeutic effect as a chip containing both an anti-inflammatory agent and an anti-bacterial agent.

1-29. (canceled)

30. An oral delivery device for the treatment of periodontal disease, said device being in a solid unit dosage form configured for insertion into a periodontal pocket of a patient, consisting of:
(a) a biodegradable pharmaceutically acceptable water-insoluble polymer in the form of a matrix;
(b) a therapeutically effective amount of at least one anti-inflammatory agent dispersed within the matrix;
(c) optionally a plasticizing agent;
(d) optionally at least one of a wetting agent, a suspending agent and a dispersing agent; and
(e) optionally an enzymatically biodegradable pharmaceutically acceptable water soluble polymer dispersed within the matrix,
wherein said biodegradable water-insoluble polymer is degradable by enzymatic degradation, physical disintegration or a combination thereof.

31. The device of claim 30 wherein said physical disintegration is by hydration and swelling of the water-insoluble polymer.

32. The device of claim 30 wherein said biodegradable water-insoluble polymer is not degradable by hydrolysis.

33. The device of claim 30 wherein said water-insoluble polymer comprises a water-soluble polymer rendered water-insoluble by the addition of a cross-linking agent in an amount sufficient to render said polymer water-insoluble, while permitting the release of said anti-inflammatory agent from said delivery device.

34. The device of claim 30 wherein said water-insoluble polymer is selected from cross-linked water-soluble protein, cellulose or cellulose derivative, starch or starch derivative, glyceryl monostearate, carbomer, PVP (polyvinylpyrrolidone), gum, acacia gum, guar gum, polyvinyl alcohol, polyhydroxyethyl metacrylate, polyhydroxymethyl metacrylate polyacrylic acid, polyacryl amide and polyethylene glycols, wherein said water-soluble protein is preferably selected from the group consisting of gelatin, collagen, albumin, an enzyme and fibrinogen, and wherein said gelatin is preferably hydrolyzed gelatin.

35. The device of claim 34 wherein said hydrolyzed gelatin may have a molecular weight in the range of 1-20 K Dalton.

36. The device of claim 33 wherein said water-soluble polymer is cross-linked by a curing process in the presence of a cross-linking agent, wherein said curing process is selected from the group consisting of heat, humidity, pressure, radiation, and the vapors of a cross-linking agent.

37. The device of claim 33 wherein said water-insoluble polymer is crosslinked in the presence of one or more of the group consisting of glutaraldehyde, formaldehyde and carbodiimide.

38. The device of claim 30 wherein said water-insoluble polymer is present at a concentration of from about 20% to about 70%.

39. The device of claim 30 wherein said plasticizing agent is selected from glycol derivatives, phthalates, citrate derivatives, benzoates, butyl or glycol esters of fatty acids, refined mineral oils, camphor, oleic acid, castor oil, corn oil and sugar alcohols, wherein said glycol derivative is preferably glycerin.

40. The device of claim 30 wherein said plasticizing agent is present at a concentration of from about 1% to about 25% (w/w).

41. The device of claim 30 wherein said anti-inflammatory agent is hydrophobic or non-water soluble.

42. The device of claim 30 wherein said anti-inflammatory agent is a non-steroidal anti-inflammatory agent (NSAID), wherein said non-steroidal anti-inflammatory agent is preferably selected from the group consisting of 3-Amino-4-hydroxybutyric Acid, Aceclofenac, Acemetacin, Acetaminosalol, Alclofenac, Alminoprofen, α-Bisabolol, Paranyline, Amfenac, Bromfenac, Benoxaprofen, Benzpiperylon, Bermoprofen, Bromosaligenin, Bucloxic Acid, Bufexamac, Bumadizon, Butibufen, Carprofen, Cinmetacin, Clidanac, Clopirac, Diclofenac, Diclofenac Sodium, Diflunisal, Ditazol, Enfenamic Acid, ε-Acetamidocaproic Acid Bendazac, Etodolac, Etofenamate, Felbinac, Fenbufen, Fenclozic Acid, Fendosal, Fenoprofen, Fentiazac, Fepradinol, Flufenamic Acid, Flunoxaprofen, Flurbiprofen, Gentisic Acid, Glucametacin, Glycol Salicylate, Ibufenac, Ibuprofen, Ibuproxam, Indomethacin, Indoprofen, Isofezolac, Isoxepac, Isoxicam, Ketoprofen, Ketorolac, Lomoxicam, Lonazola, Lonazolac, Loxoprofen, Meclofenamic Acid, Mefenamic Acid, Meloxicam, Mesalamine, Metiazinic Acid, Mofebutazone, Mofezolac, Naproxen, Niflumic Acid, Olsalazine, Oxaceprol, Oxametacine, Oxaprozin, Oxicams, Oxyphenbutazone, Paranyline, Parsalmide, Perisoxal, Phenyl Salicylate, Pirazolac, Piroxicam, Pirprofen, Pranoprofen, Proprionic Acids, Protizinic Acid, Salacetamide, Salicilic Acid, Salicylamide O-Acetic Acid, Salicylsulfuric Acid, Salsalate, Sulfasalazine, Sulindac, Suprofen, Suxibuzone, Talniflumate, Tenoxicam, Terofenamate, Tiaprofenic Acid, Tiaramide, Tinoridine, Tolfenamic Acid, Tolmetin, Tropesin, Xenbucin, Ximoprofen, Zaltoprofen, Zileuton and Zomepirac.

43. The device of claim 42 wherein said anti-inflammatory agent is flurbiprofen.

44. The device of claim 30 in the form of a film, pellet, granule or cylinder, wherein said film is preferably from about 3 to about 6 mm in length and from about 1 to about 5 mm in width and from about 0.01 to about 1.0 mm in thickness.

45. The device of claim 30 wherein said anti-inflammatory agent and said water-insoluble polymer are present at a relative weight ratio which ranges from about 2:1 to about 1:3.

46. The device of claim 30 wherein said plasticizing agent and said polymer are present at a relative weight ratio which ranges from about 1:10 to about 1:2.

47. A periodontal implant comprising the device of claim 30.

48. A method for the treatment of periodontal disease comprising administering to a periodontal pocket of a patient in need of such treatment the delivery device of claim 30.

49. The method of claim 48 wherein said treatment is an adjunct treatment to periodontal surgery, wherein said device is inserted into a periodontal pocket before and/or after the periodontal surgery.
